(19) United States
(12) Patent Application Publication
(10) Pub. No.: US 2008/0200934 A1
(43) Pub. Date: Aug. 21, 2008

(54) Surgical devices and methods using magnetic force to form an anastomosis

(76) Inventor: William D. Fox, New Richmond, OH (US)

Correspondence Address: KIRKPATRICK & LOCKHART PRESTON GATES ELLIS LLP, 535 SMITHFIELD STREET, PITTSBURGH, PA, 15222, US

(21) Appl. No.: 11/706,685

(22) Filed: Feb. 15, 2007

(51) Int. Cl. A61B 17/08

(52) U.S. Cl. 606/153

(57) ABSTRACT

A method for forming an anastomosis between first and second organs in a patient using a hollow receptacle that is inflatable with magnetic material. The method may include forming openings through the first and second organs utilizing a hole-forming instrument inserted into the organs through a natural orifice in the patient. The hollow receptacle may be supported on a catheter assembly that is also inserted through the patient's natural orifice and through the openings in the first and second organs and is positioned within the second organ. The hollow receptacle is then inflated with magnetic material and magnetic force is applied within the force organ to draw the inflated receptacle toward the first organ such that the inflated receptacle retains the second organ in sealing contact with the first organ while maintaining the alignment between the first and second openings to create an anastomosis between the first and second organs.

### CROSS-REFERENCE TO RELATED APPLICATIONS

The present application is related to the following commonly-owned U.S. patent application filed on even date herewith, entitled “Surgical Devices and Methods For Forming An Anastomosis Between Organs By Gaining Access Thereto Through a Natural Orifice in the Body” to Gregory J. Bakos and William D. Fox, (END6103USNP/KLG No. 070031) the disclosure of which is hereby incorporated by reference in its entirety.

### FIELD OF THE INVENTION

The present invention relates, in general, to surgical devices for forming an anastomosis between organs and, more particularly, to devices that can be inserted through a natural orifice in the body and used to form an anastomosis between various gastrointestinal organs.

### BACKGROUND OF THE INVENTION

Access to the abdominal cavity may, from time to time, be required for diagnostic and therapeutic endeavors for a variety of medical and surgical diseases. Historically, abdominal access has required a formal laparotomy to provide adequate exposure. Such procedures which require incisions to be made in the abdomen are not particularly well-suited for patients that may have extensive abdominal scarring from previous procedures, those persons who are morbidly obese, those individuals with abdominal wall infection, and those patients with diminished abdominal wall integrity, such as patients with bums and skin grafting. Other patients simply do not want to have a scar if it can be avoided.

In the past, such surgical procedures were also employed to address various problems occurring in the jejunum (a portion of the small intestine). For example, such procedures were commonly employed to address blockages or strictures in the jejunum or to address diseases occurring therein. In some situations, it becomes necessary to create a Gastro-Jejunostomy—an anastomosis between the stomach and the jejunum. In addition to suffering from the above-mentioned limitations, current laparoscopic and endoscopic surgical techniques also fail to provide a convenient way for inserting a distal mass and are generally incapable of applying sufficient mass and force to effect a clinically acceptable compression anastomosis.

Consequently a significant need exists for an alternative to conventional surgery that eliminates abdominal incisions and incision-related complications by combining endoscopic and laparoscopic techniques to diagnose and treat abdominal pathology.

There is a further need for a surgical device that can be introduced into the stomach through the mouth and used to form a clinically acceptable compression anastomosis between the stomach and the jejunum.

The foregoing discussion is intended only to illustrate some of the shortcomings present in the field of the invention at the time, and should not be taken as a disavowal of claim scope.

### SUMMARY

In one aspect of the invention, there is provided a method for forming an anastomosis between first and second organs in a patient. The method may comprise forming a first opening in the first organ and forming a second opening in the second organ. Thereafter, an inflatable receptacle is inserted into the second organ. A magnetic material is injected into the inflatable receptacle to inflate the receptacle within the second organ. Magnetic force is used to draw the inflated receptacle toward the first organ such that the inflated receptacle retains the second organ in sealing contact with the first organ to create an anastomosis between the first and second organs.

In another general aspect of various embodiments of the present invention there is provided a method for forming an anastomosis between first and second organs in a patient. In one embodiment, the method may include inserting a hole-forming instrument through a natural orifice in the patient to form aligned holes through the first and second organs. The hole-forming instrument may then be withdrawn out through the natural orifice. A catheter assembly supporting an inflatable receptacle thereon is inserted through the natural orifice and the aligned holes to position the inflatable receptacle within the second organ. Magnetic material is then introduced into the inflatable receptacle. Magnetic force is used to attract the receptacle inflated with magnetic material into sealing engagement with a corresponding portion of the first organ while maintaining the alignment between the first and second holes to create an anastomosis between the first and second organs. Thereafter, a proximal portion of the catheter assembly may be withdrawn out through the natural orifice.

In still another general aspect of various embodiments of the present invention there is provided a surgical instrument for creating an anastomosis between two organs. In various embodiments, the instrument comprises a catheter assembly that has a distal end portion and a proximal end portion and is configured for insertion through a first organ into a second organ adjacent the first organ. The catheter assembly may further have a supply lumen extending therethrough that contains magnetic material therein. A hollow inflatable receptacle extends around a portion of the distal end portion of the catheter assembly. The hollow inflatable receptacle defines a substantially fluid-tight hollow space that communicates with the supply lumen for receiving the magnetic material therein upon application of a pressure medium to the supply lumen. A magnet is movably receivable on a proximal portion of the catheter assembly that is located within the first organ such that the magnet may be positioned within the first organ to magnetically attract the receptacle inflated with the magnetic material thereto and thereby draw a portion of the second organ containing the inflated receptacle toward a corresponding portion of the first organ into sealing engagement therewith.

These and other objects and advantages of the present invention shall be made apparent from the accompanying drawings and the description thereof.

### BRIEF DESCRIPTION OF THE FIGURES

The accompanying drawings, which are incorporated in and constitute a part of this specification, illustrate embodiments of the invention, and, together with the general description of the invention given above, and the detailed description of the embodiments given below, serve to explain various principles of the present invention.

FIG. 1 is a diagrammatical view illustrating the use of one embodiment of the surgical instrument of the present invention inserted through a patient's mouth and esophagus to create an anastomosis between the patient's stomach and jejunum;

FIG. 2 is a perspective view of a distal portion of a surgical instrument embodiment of the present invention;

FIG. 3 is a cross-sectional view of the distal portion of the surgical instrument embodiment depicted in FIG. 2, with a guide wire extending therethrough shown in solid form;

FIG. 4 is a cross-sectional view of a portion of a distal catheter assembly coupled to a portion of a proximal catheter assembly of various embodiments of the present invention;

FIG. 5 is another cross-sectional view of the distal catheter assembly and proximal catheter assembly of FIG. 4 illustrating the insertion of a guide wire therethrough;

FIG. 6 is another cross-sectional view of the distal catheter assembly and proximal catheter assembly of FIGS. 4 and 5 illustrating the guide wire therethrough and the injection of magnetic material through the instrument;

FIG. 7 is another cross-sectional view of the distal catheter assembly and proximal catheter assembly of FIGS. 4-6 illustrating a portion of the filed receptacle and positioning of the magnet of various embodiments of the present invention;

FIG. 8 is a perspective view of the distal catheter assembly with the receptacle portion thereof in an inflated/expanded state within a portion of the jejunum and of the proximal catheter assembly supporting a magnet thereon within the stomach;

FIG. 9 is diagrammatical view of a patient's esophagus, stomach, and jejunum with a conventional gastroscope inserted into the stomach through the esophagus;

FIG. 10 is another diagrammatical view of the patient's esophagus, stomach, and jejunum as depicted in FIG. 9 with a fiber scope inserted through a working channel in the gastroscope;

FIG. 11 is another diagrammatical view of the patient's esophagus, stomach, and jejunum depicted in FIG. 10 and wherein a needle knife or hot wire has additionally been inserted through the fiber scope to puncture through a portion of the stomach wall;

FIG. 12 is another diagrammatical view of the patient's esophagus, stomach, and jejunum depicted in FIG. 11 wherein the needle knife has punctured a portion of the stomach wall and jejunum and wherein a guide wire has been inserted through the needle knife into the jejunum;

FIG. 13 is another diagrammatical view of the patient's esophagus, stomach, and jejunum illustrated in FIG. 12 after the fiber scope and needle knife have been withdrawn;

FIG. 14 is another diagrammatical view of the patient's esophagus, stomach, and jejunum illustrated in FIG. 13 after one surgical instrument embodiment has been inserted through a working channel in the gastroscope into the jejunum along the guide wire;

FIG. 15 is another diagrammatical view of the patient's esophagus, stomach, and jejunum illustrated in FIG. 14 after the receptacle portion of the surgical instrument has been inflated or expanded with magnetic material;

FIG. 16 is another diagrammatical view of the patient's esophagus, stomach, and jejunum illustrated in FIG. 14 after the receptacle portion of the surgical instrument has been inflated or expanded with magnetic material;

FIG. 17 is another diagrammatical view of the patient's esophagus, stomach, and jejunum illustrated in FIG. 16 after a magnetic has been moved along the instrument into position adjacent the stomach wall;

FIG. 18 is another diagrammatical view of the patient's esophagus, stomach, and jejunum illustrated in FIG. 17 after the instrument has been moved in a proximal to draw the wall of the jejunum into contact with the stomach wall;

FIG. 19 is a cross-sectional view of a portion of the proximal catheter assembly after it has been decoupled from a portion of the distal catheter assembly of various embodiments of the present invention;

FIG. 20 is another diagrammatical view of the patient's esophagus, stomach, and jejunum illustrated in FIG. 18 after the proximal catheter assembly has been decoupled from the distal catheter assembly and after the pig tail stent/positioner has been installed to retain the anastomosis assembly in position;

FIG. 21 is another diagrammatical view of the patient's esophagus, stomach, and jejunum illustrated in FIG. 20 illustrating passing of the anastomosis assembly and formation of a natural fistula between the stomach and jejunum; and

FIG. 22 is a cross-sectional view of another catheter assembly embodiment of the present invention illustrating a portion of the filed receptacle and magnet thereon.

### DETAILED DESCRIPTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the various embodiments of the present invention is defined solely by the claims. The features illustrated or described in connection with one exemplary embodiment may be combined with the features of other embodiments. Such modifications and variations are intended to be included within the scope of the present invention.

The present invention generally provides methods and devices for creating a Gastro-Jejunostomy via natural orifice procedures combined with a transorgan approach. While various exemplary embodiments are described herein for creating an anastomosis between the stomach and the small intestine by accessing those organs through the patient's mouth and esophagus, those of ordinary skill in the art will readily appreciate that unique and novel aspects of various embodiments of the present invention could successfully be employed in connection with forming anastomosis between other organs by gaining access thereto through other natural orifices such as, for example, the anus, the vagina, etc. without departing from the spirit and scope of the present invention..

It will be appreciated that the terms “proximal” and “distal” are used herein with reference to a clinician manipulating and end of the instrument 20 that protrudes out of the patient's mouth. The term “proximal” referring to the portion closest to the surgeon and the term “distal” referring to the portion located away from the surgeon. It will be further appreciated that for convenience and clarity, spatial terms such as “vertical”, “horizontal”, “up” and “down” may be used herein with respect to the drawings. However, surgical instruments are used in many orientations and positions, and these terms are not intended to be limiting and absolute.

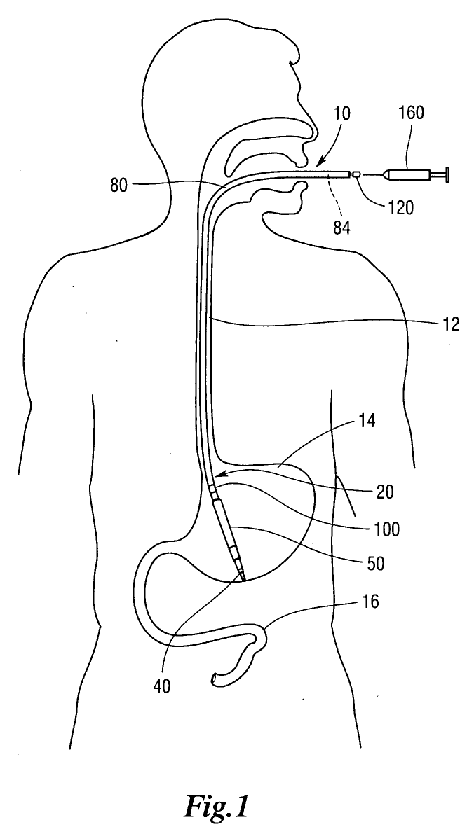

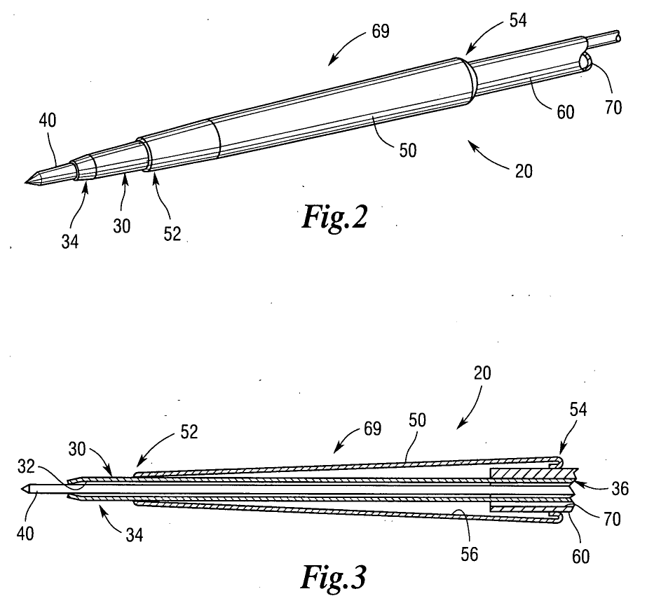

FIG. 1 illustrates, in general form, one exemplary surgical instrument 20 of the present invention that can be inserted through a natural orifice such as the mouth 10 and esophagus 12 into the stomach 14 to establish an anastomosis between the stomach 14 and the jejunum 16. As can be seen in FIGS. 2-6, the surgical instrument 20 may comprise a distal inner catheter segment 30 that contains a first distal lumen 32 which is adapted to accommodate a guide wire 40, the purpose of which will be discussed in further detail below. In various embodiments, the distal inner catheter 30 may be fabricated from, for example, nylon or other plastic materials. The first distal lumen 32 may be sized to accommodate a 0.035 inch (0.89 mm) diameter guide wire. Other suitable guide wire arrangements could also be successfully employed.

In various embodiments, a hollow receptacle 50 is provided around a portion of the distal inner catheter segment 30. The hollow receptacle 50 may comprise an expandable balloon, pouch or bag that extends around, and is attached at its distal end portion 52 to the distal inner catheter segment 30 and at its proximal end portion 54 to a distal outer catheter segment 60 to define a hollow interior space generally designated as 56. See FIG. 3. As can be seen in FIG. 3, a distal end portion 34 of the distal inner catheter segment 30 extends through the hollow receptacle 50 and protrudes outward from the distal end 52 thereof. The distal inner catheter segment 30 extends through the hollow receptacle 50 and is received within the distal outer catheter segment 60. The distal outer segment 60 may be fabricated from, for example, nylon or other plastic materials. In various embodiments, the hollow expandable receptacle 50 may be fabricated from silicone or latex rubber, or a variety of other elastomers and be attached to the distal inner catheter segment 30 and the distal outer catheter segment 60 by, for example, suitable adhesive such as cyanoacrylate or epoxy glues, heat seal or light activated adhesives such that a substantially fluid tight seal is established between the distal inner catheter segment 30, the distal outer catheter segment 60 and the hollow receptacle 50. In other embodiments, the hollow receptacle 50 may be fabricated from a material that is not substantially expandable, such as nylon, polyester or PET, or a variety of other polymers, but nevertheless is sized to inflate into a desired shape as will be further described below. The distal inner catheter segment 30, the hollow receptacle 50 and the distal outer catheter segment 60 are generally referred to herein collectively as the distal catheter assembly 69.

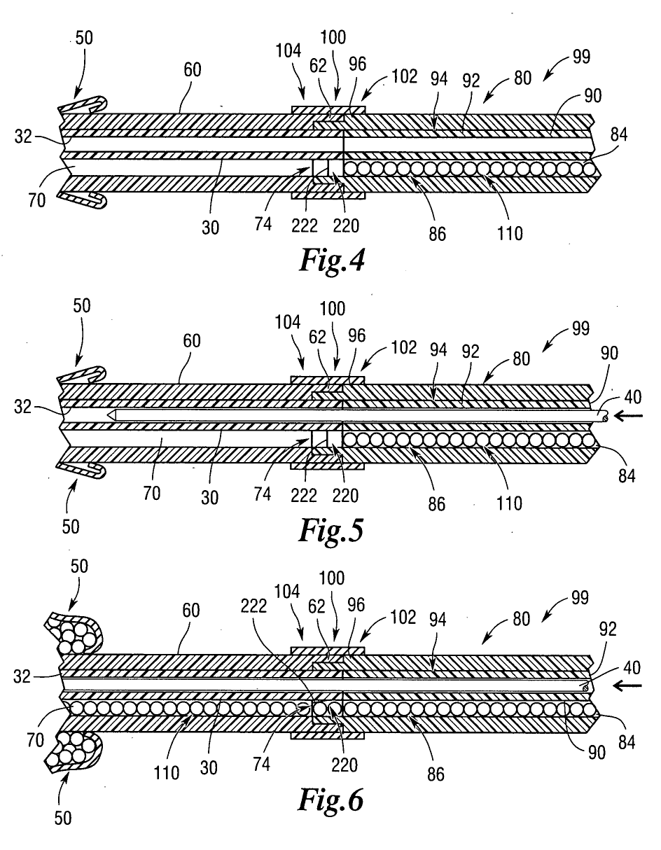

In various embodiments, a proximal portion 36 of the distal inner catheter segment 30 is received in the distal outer catheter segment 60. See FIG. 3. The distal outer catheter segment 60 also has a magnetic material supply lumen 70 formed therein that opens into or is in fluid communication with, the hollow interior 56 inside of the hollow receptacle 50. As used herein, the term “fluid communication” means that the elements are coupled together with an appropriate lumen, supply passage, line or other means to permit the passage of fluid (air, water, saline, etc.) therebetween. In the embodiment depicted in FIGS. 4-6, the proximal end 62 of the distal outer catheter segment 60 is configured to mate with a distal end 82 of a proximal catheter segment 80. In alternative embodiments, the distal outer catheter segment 60 is one piece and is sufficiently long enough to extend from the surgical site, through the esophagus and out through the patient's mouth. When employing that embodiment, upon completion of the anastomosis procedure, the distal outer catheter segment must be severed adjacent the anastomosis to enable the remaining portion thereof to be removed from the patient as will be further described below.

In the embodiment depicted in FIGS. 4-6, the proximal outer catheter segment 80 supports a proximal inner catheter segment 90 therein that has a proximal lumen 92 therein that is oriented to mate with the distal lumen 32 in the distal inner catheter segment 30 when the distal outer catheter segment 60 is coupled to the proximal outer catheter segment 80. As can be seen in FIGS. 4-6, when the distal inner catheter segment 30 is oriented in end-to-end fashion with the proximal inner catheter segment 90, the lumens 32 and 92 cooperate to form a substantially continuous lumen generally designated as 94 for receiving the guide wire 40 therethrough. Likewise, the proximal outer catheter portion 80 has a second magnetic particle supply lumen 84 formed therein that is oriented to mate with the first magnetic material supply lumen 70 in the distal outer catheter segment 60 to form a substantially continuous magnetic particle supply lumen generally designated as 86 when the distal outer catheter segment 60 is coupled to the proximal outer catheter segment 80. In various embodiments, the distal outer catheter segment 60 is coupled to the proximal outer catheter segment 80 by a coupling tube. 100. The proximal end 102 of the coupling tube 100 is permanently affixed to the distal end 96 of the proximal outer catheter segment 80 by, for example, adhesive. The distal end 104 of the coupling tube 104 slides over the proximal end 62 of the distal outer catheter segment 60 and is retained in position by virtue of a frictional fit therebetween. As the present Detailed Description proceeds, the frictional fit between the coupling tube 100 and the proximal end 62 of the distal outer catheter segment 60 is of a sufficient magnitude so as to maintain a substantially fluid tight seal therebetween when magnetic material 110 is injected under pressure though the substantially continuous magnetic material supply lumen 86, yet enable the coupling tube 100 to be disengaged from the distal outer catheter segment 60 after the procedure has been completed. The proximal outer catheter segment, the proximal inner catheter segment 90 and connector tube 100 is generally referred to herein as a proximal catheter assembly 99.

In various embodiments, prior to commencing the procedure, the second magnetic material supply lumen 84 in the proximal outer catheter segment 80 contains magnetic material 110 which may comprise, for example, magnetic beads, magnetic balls, magnetic pellets, magnetic dust, magnetic particles or a combination of two or more of such materials. In one embodiment, for example, 1 mm diameter beads fabricated from 400 Series Stainless Steel are employed. As can be seen in FIG. 1, a conventional luer fitting 120 can be attached to (e.g., is in fluid communication with) the second magnetic material supply lumen 84 in the proximal outer catheter segment 80. The luer fitting 120 enables the surgeon to inject a pressure medium (saline solution, air, etc.) into the second magnetic particle supply lumen 84 to force the magnetic material 110 contained therein through the substantially continuous magnetic material supply lumen 86 and into the hollow receptacle 50. The saline solution and the magnetic material 110 form a slurry of magnetic material. In other embodiments, the surgeon may simply inject air into the second magnetic material supply lumen 84 through the luer fitting 120 to force the magnetic material 110 into the hollow receptacle 50.

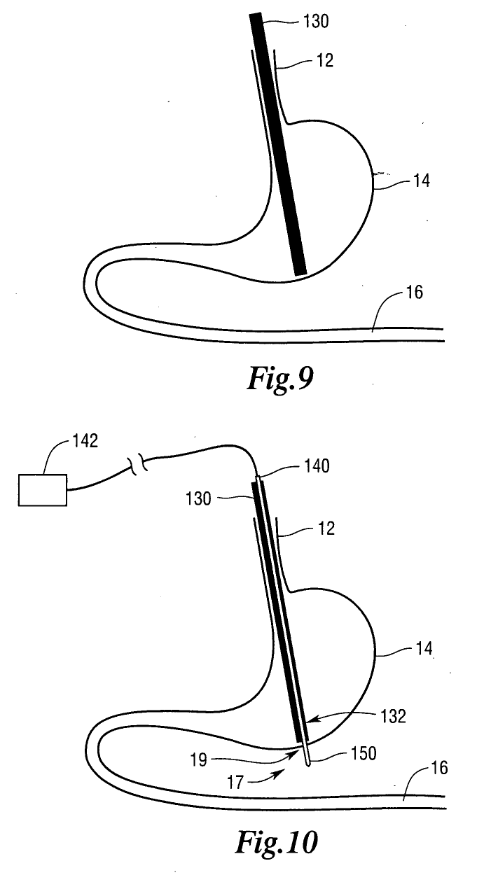

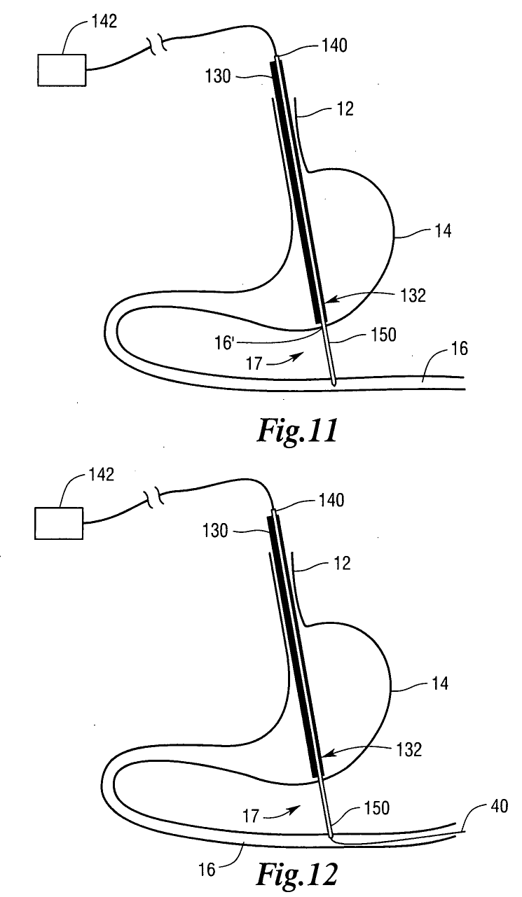

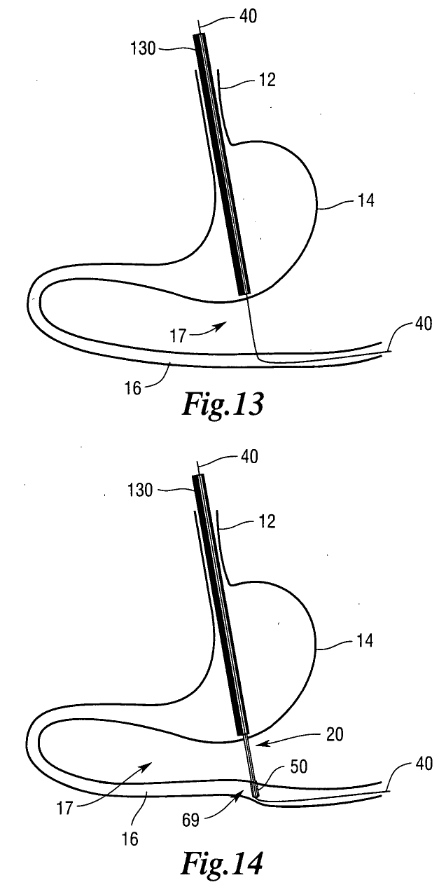

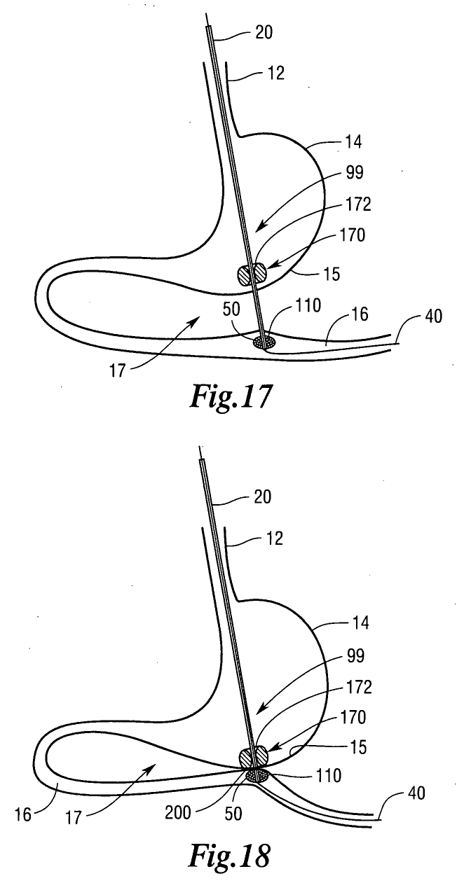

Use of the surgical instrument 20 will now be described with reference to FIGS. 1 and 6-21. To begin one method of the present invention for forming an anastomosis between two organs, the surgeon may insert a conventional gastroscope 130 through the patient's mouth 10 (FIG. 1) and esophagus 12 into the patient's stomach 14. See FIG. 9. The construction and operation of such gastroscopes are known in the art and thus will not be discussed in detail herein. Such gastroscopes generally have at least one working channel 132 therein through which additional instruments may be inserted. In one exemplary method, the surgeon inserts a conventional fiber optic scope 140 (FIG. 10) that is coupled to a display 142 which enables the surgeon to view the surgical field. Once the surgeon has maneuvered the gastroscope 130 to a desired position, a hole-forming instrument such as a needle knife 150 or radio frequency based wire is inserted through a working channel in the gastroscope to form a hole or opening 19 into the stomach wall 15. See FIG. 10. Then, using the fiber optic scope 140 to view the peritoneum 17 and locate a desired point on the jejunum 16, the surgeon uses the hole-forming needle knife 150 or radio frequency based wire to form a hole 18 through the wall 16′ of the jejunum 16 at the desired location. See FIG. 11. After the jejunum 16 has been entered by the needle knife 150 or radio frequency based wire, the guide wire 40 is inserted through a lumen (not shown) in the needle knife 150 or radio frequency based wire and into the jejunum 16 as illustrated in FIG. 12. The fiber optic scope 140 along with the needle knife 150 or radio frequency based wire are then withdrawn from the working channel 132 in the gastroscope while leaving the guide wire 40 in the jejunum 16. See FIG. 13.

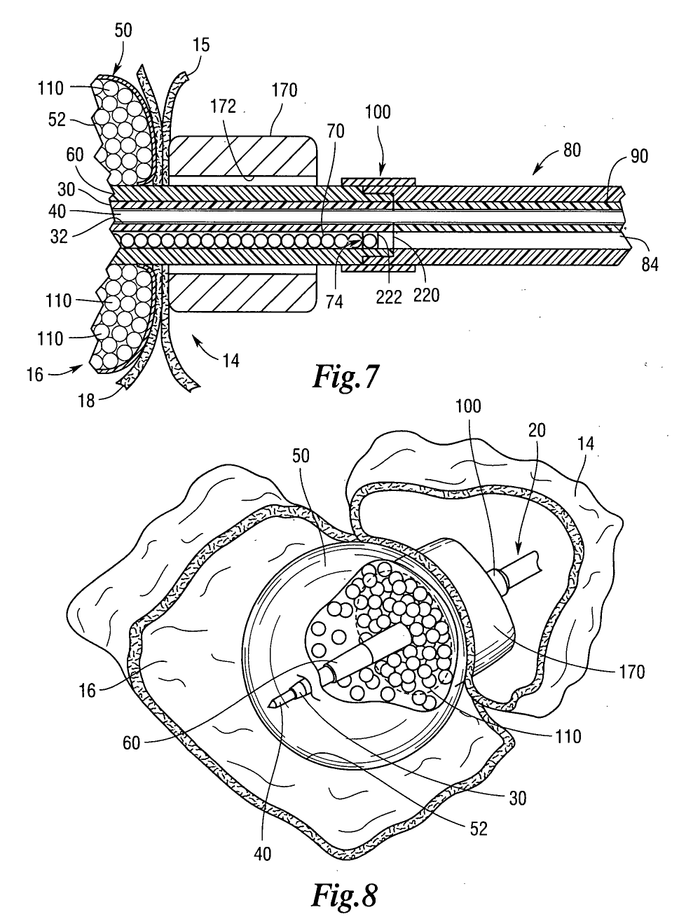

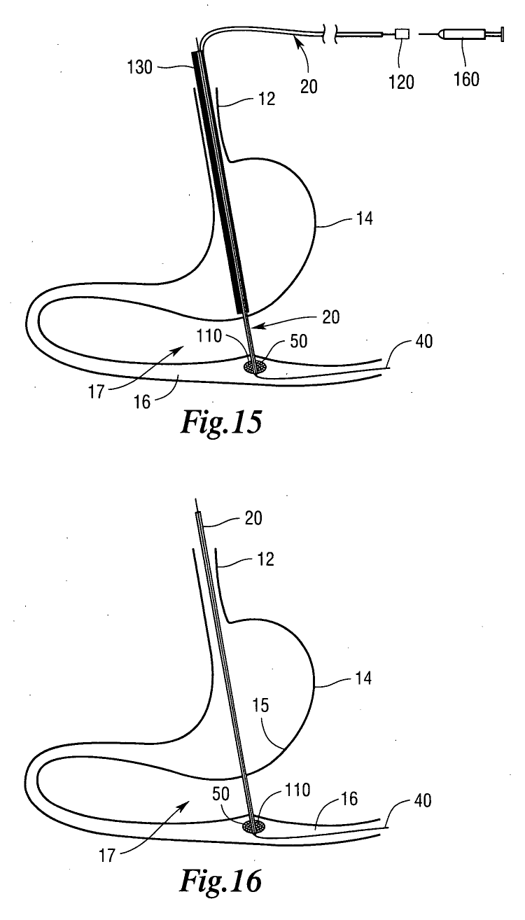

After the fiber scope 140 and the needle knife 150 have been withdrawn, the surgical instrument 20 may then be inserted through the working channel 132 in the gastroscope 130 and essentially threaded onto the guide wire 40 to guide the distal catheter assembly 69 of the instrument 20 into the jejunum 16 as shown in FIG. 14. After the hollow receptacle 50 has been positioned in the jejunum 16, the magnetic material 110 can be injected into the hollow receptacle 50 by injecting a saline solution or air, for example, contained in a syringe 160 in through the luer fitting 120 that is coupled to the second magnetic material supply lumen 84. See FIG. 15. As the saline or air is injected into the lumen 84, the slurry of magnetic material 110 is forced into the hollow receptacle 50 causing it to expand/inflate. See FIGS. 7, 8 and 15. The gastroscope 130 is then withdrawn as illustrated in FIG. 16.

After the gastroscope 130 has been withdrawn, magnetic force is then applied to draw the inflated receptacle 50 toward the stomach 14 such that the inflated receptacle 50 retains the jejunum 16 in sealing contact with the stomach while maintaining the alignment between the opening in the stomach and the jejunum to create an anastomosis between the stomach 14 and the jejunum 16. The magnetic force may be applied through a proximal magnet 170 that is slid over the proximal catheter assembly 99 and positioned against the stomach wall 15. As can be seen in FIGS. 7, 17, and 18, the magnet 170 is somewhat donut-shaped and has a central passage 172 therethrough that is sized to enable the magnet 170 to slide over the connector tube 100 into position. In various embodiments, the magnet 170 may be fabricated from magnetic material such as Neodymium that is shrouded with plastic or rubber. To assist with the positioning of the magnet 170 against the stomach wall 15, the surgeon may thread a hollow pusher tube (not shown) or other suitable device over the proximal catheter assembly 99 to push the magnet 170 therealong. After the magnet 170 has been positioned against the stomach wall 15, the instrument 20 is moved proximally to draw the expanded receptacle 50 toward the stomach wall 15. As the surgeon draws the expanded receptacle 50 toward the stomach wall 15, the receptacle 50 draws a portion of the jejunum wall 18 into contact with the stomach wall 15. See FIGS. 8 and 18. The distal inner catheter segment 30, receptacle 50 and magnet 170 cooperate to form an anastomosis assembly, generally designated as 200.

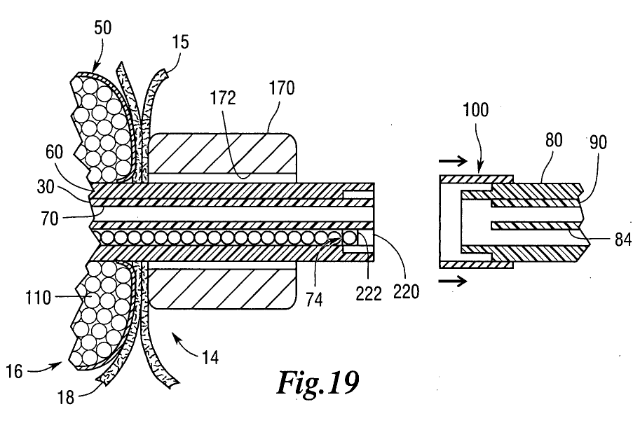

Once the receptacle 50 and magnet 170 have been snugged against the jejunum wall 18 and stomach wall 15, respectively, the magnet material 110 in the receptacle 50 is attracted to the magnet 170 to retain the walls 15, 17 in that position. Thereafter, a commercially available hollow pigtail type stent/positioner 210 is threaded onto the guide wire 40 and advanced therealong through the substantially continuous lumen 94 formed by the lumens 32 and 92 in the distal inner catheter segment 30 and the proximal inner catheter segment 90, respectively. The surgeon may advance the pigtail stent/positioner 210 along the guide wire 40 by means of a hollow tube (not shown) sized to be movably received in the lumen 94. The pigtail stent/positioner 210 is moved to the position illustrated in FIG. 19 such that the distal end 212 thereof protrudes distally out of the receptacle 50 through the distal inner catheter segment 30.

After the pigtail stent 210 has been moved into position, the guide wire 40 and the proximal catheter assembly 99 are withdrawn. As the surgeon applies a withdrawal force to the proximal catheter assembly 99, the connector tube 100 slides off of the proximal end 62 of the distal outer catheter segment 60 leaving the distal outer catheter segment 60 behind. Those of ordinary skill in the art will understand that the inner diameter of the connector tube 100 is sized relative to the outer diameter of the proximal end 62 of the distal outer catheter segment 60 to create a sufficient amount of friction therebetween to maintain a substantially fluid-tight seal therebetween as the magnetic material 110 is injected into the receptacle 50, yet not be so great as to prevent the connector tube 100 from sliding off of the proximal end 62 of the distal outer catheter segment 60 without undesirably moving the anastomosis assembly 200 out of position.

Also in various embodiments, to prevent the magnetic material 110 from back flowing out through the first magnetic particle supply lumen 70 after the proximal catheter assembly 99 has been decoupled from the distal catheter assembly 69, a check valve 220 may be provided in the proximal end 74 of the lumen 70. See FIGS. 4-7 and 19. The check valve 220 may have a flapper valve 222 therein that permits flow of the magnetic material 110 in the distal direction, but moves to a closed position when the material 110 attempts to flow in the proximal direction. Other check valve arrangements could be employed to prevent backflow of the magnetic material 110 into the stomach 14.

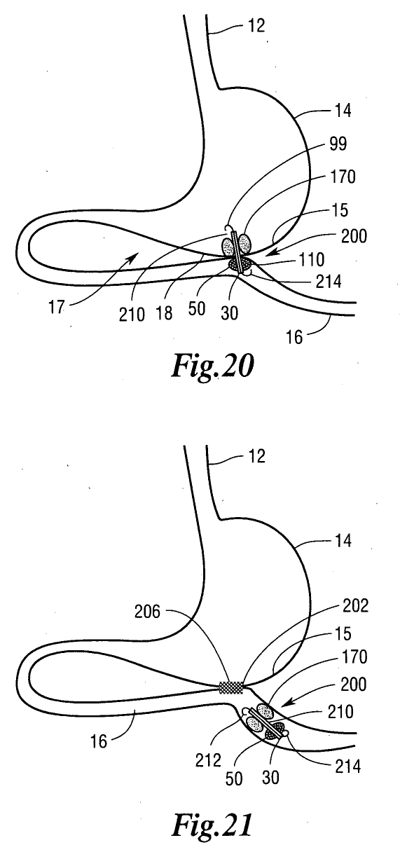

After the proximal catheter assembly 99 has been decoupled from the distal catheter assembly 69 and withdrawn from the patient and the guide wire 40 has also been withdrawn, the proximal end 212 and the distal end 214 of the pigtail stent 210 assume the “pigtail-like” shape to retain the anastomosis assembly 200 in position. See FIG. 20. After a period of time, such as, seven to ten days, for example, the anastomosis assembly 200 can pass naturally out through the small intestine 16 leaving a permanent anastomosis and fistula 202 which may be optionally enlarged with a stent 206. See FIG. 21.

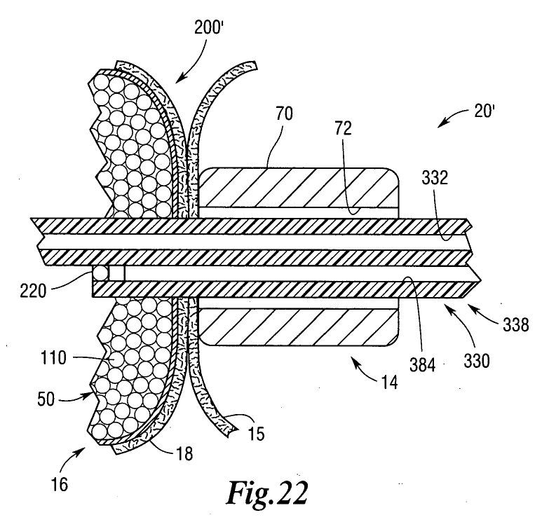

The above-described embodiments employ a surgical instrument 20 that includes a distal catheter assembly 69 and a proximal catheter assembly 99. After the anastomosis assembly 200 is formed, the proximal catheter assembly 99 is decoupled from the distal catheter assembly 69 and withdrawn from the patient leaving the anastomosis assembly 200 in position. In alternative embodiments, such as the surgical instrument 20′ shown in FIG. 22, the surgical instrument 20′ may be fabricated from a single catheter 330 that has dual lumens 332 and 384 therein. Lumen 332 is sized to receive the guide wire 40 and pig tail stent 210 and lumen 384 is sized to supply the magnetic material/slurry to the receptacle 50. In this embodiment, a check valve 220, located inside of the substantially hollow receptacle 50 is coupled to the lumen 384 to permit the magnetic material 110 to flow into the receptacle 50, but prevent the material 110 from flowing back through the lumen 384. This surgical instrument 20′ embodiment is essentially employed in the same manner as described above with respect to instrument 20, except that, a proximal portion 338 of the catheter 330 must be cut from the anastomosis assembly 200′. This may be accomplished through use of a catheter cutting device or hot wire device that may be fed down a working passage of an endoscope threaded onto the catheter.

While the present invention has been illustrated by description of several embodiments and while the illustrative embodiments have been described in considerable detail, it is not the intention of the applicant to restrict or in any way limit the scope of the appended claims to such detail. Additional advantages and modifications may readily appear to those skilled in the art. Those of ordinary skill in the art will readily appreciate the different advantages provided by these various embodiments. While the various surgical instruments have been herein described in connection with the formation of a Gastro-Jejunostomy through a patient's mouth, those of ordinary skill in the art will readily appreciate that the unique and novel features of the various embodiments of the present invention may be effectively employed in connection with forming an anastomosis between other organs which may be accessed through other natural orifices in the patient. In addition, it is conceivable that the various embodiments of the present invention could have utility in some laparoscopic surgical procedures and therapies.

While several embodiments of the invention have been described, it should be apparent, however, that various modifications, alterations and adaptations to those embodiments may occur to persons skilled in the art with the attainment of some or all of the advantages of the invention. For example, according to various embodiments, a single component may be replaced by multiple components, and multiple components may be replaced by a single component, to perform a given function or functions. This application is therefore intended to cover all such modifications, alterations and adaptations without departing from the scope and spirit of the disclosed invention as defined by the appended claims.

The devices disclosed herein can be designed to be disposed of after a single use, or they can be designed to be used multiple times. In either case, however, the device can be reconditioned for reuse after at least one use. Reconditioning can include an combination of the steps of disassembly of the device, followed by cleaning or replacement of particular pieces, and subsequent reassembly. In particular, the device can be disassembled, and any number of particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those of ordinary skill in the art will appreciate that the reconditioning of a device can utilize a variety of different techniques for disassembly, cleaning/replacement, and reassembly. Use of such techniques, and the resulting reconditioned device, are all within the scope of the present application.

Preferably, the invention described herein will be processed before surgery. First a new or used instrument is obtained and, if necessary, cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or higher energy electrons. The radiation kills bacteria on the instrument and in the container. The sterilized instrument can then be stored in the sterile container. The sealed container keeps the instrument sterile until it is opened in the medical facility.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

The invention which is intended to be protected is not to be construed as limited to the particular embodiments disclosed. The embodiments are therefore to be regarded as illustrative rather than restrictive. Variations and changes may be made by others without departing from the spirit of the present invention. Accordingly, it is expressly intended that all such equivalents, variations and changes which fall within the spirit and scope of the present invention as defined in the claims be embraced thereby.

What is claimed is:

1. A method for forming an anastomosis between first and second organs in a patient, comprising:
forming a first opening in the first organ;
forming a second opening in the second organ;
inserting an inflatable receptacle into the second organ;
injecting a magnetic material into the inflatable receptacle to inflate the receptacle within the second organ;
positioning the second organ-adjacent to the first organ; and
using magnetic force to draw the inflated receptacle toward the first organ such that the inflated receptacle retains the second organ in sealing contact with the first organ while creating an anastomosis between the first and second organs.

2. A method according to claim 1 wherein said forming a first opening comprises:
passing a hole-forming instrument through a natural orifice in the patient into the first organ;
creating the first opening in the first organ using the hole-forming instrument.

3. A method according to claim 2 wherein said forming a second opening comprises:
passing the hole-forming instrument through the first opening; and
creating the second opening in the second organ using the hole-forming instrument.

4. A method according to claim 3 further comprising passing a guide member through the natural orifice into the first and second openings.

5. A method according to claim 4 wherein said inserting an inflatable receptacle into the second organ comprises:
providing a catheter assembly having a distal portion and a proximal portion and a guide lumen extending therethrough wherein the inflatable receptacle is supported on the distal portion and is in fluid communication with a supply lumen in the catheter assembly;
inserting a proximal end of the guide member protruding from the natural orifice into the guide lumen in the catheter assembly; and
moving the catheter on the guide member until the distal portion of the catheter assembly supporting the hollow receptacle is located within the second organ and the proximal portion of the catheter is accessible through the natural orifice.

6. A method according to claim 5 wherein said injecting a magnetic material into the inflatable receptacle comprises:
providing the magnetic material within the supply lumen; and
forcing the magnetic material through the supply lumen into the inflatable receptacle.

7. A method according to claim 6 wherein said forcing comprises injecting a medium under pressure into the supply lumen to cause the magnetic material to enter the inflatable receptacle.

8. A method according to claim 6 wherein said using magnetic force to draw the inflated receptacle toward the first organ comprises advancing a magnet on the catheter assembly into a position on the distal portion of the catheter assembly adjacent the inflated receptacle located in the second organ such that said magnet draws the inflated receptacle toward the magnet and traps portions of the first and second organs therebetween while retaining the first opening and second opening in substantial alignment.

9. A method according to claim 8 further comprising installing a retention member through the distal portion of the catheter assembly to temporarily retain the distal portion of the catheter assembly within the first and second openings.

10. A method according to claim 9 further comprising:
withdrawing the guide member out through the natural orifice;
detaching the proximal portion of the catheter assembly from the distal portion of the catheter assembly such that the distal portion of the catheter assembly, magnet, inflated receptacle and retention member collectively form an anastomosis assembly temporarily supported within the aligned first and second openings; and
withdrawing the detached proximal portion of the catheter assembly through the natural orifice.

11. A method according to claim 10 further comprising passing the anastomosis assembly out through the second organ after a period of time.

12. A method according to claim 2 wherein the natural orifice comprises the patient's mouth and wherein the first organ comprises the patient's stomach and wherein the second organ comprises the patient's jejunum.

13. A method for forming an anastomosis between first and second organs in a patient, comprising:
inserting a hole-forming instrument through a natural orifice in the patient to form aligned holes through the first and second organs;
withdrawing the hole-forming instrument out through the natural orifice;
inserting a catheter assembly supporting an inflatable receptacle thereon through the natural orifice and the aligned holes to position the inflatable receptacle within the second organ;
injecting the inflatable receptacle with magnetic material;
using magnetic force to attract the receptacle inflated with magnetic material into sealing engagement with a corresponding portion of the first organ while maintaining the alignment between the first and second holes to create an anastomosis between the first and second organs; and
withdrawing a proximal portion of the catheter assembly out through the natural orifice.

14. A surgical instrument for creating an anastomosis between two organs, comprising:
a catheter assembly having a distal end portion and a proximal end portion and configured for insertion through a first organ into a second organ adjacent the first organ, said catheter assembly further having a supply lumen extending therethrough;
magnetic material supported within said supply lumen;
an inflatable receptacle extending around a portion of said distal end portion of said catheter assembly and defining a substantially fluid-tight hollow space therebetween, wherein said space is in communication with said supply lumen and is configured to receive said magnetic material therein upon application of a pressure medium to said supply lumen; and
a magnet movably receivable on a proximal portion of said catheter assembly located within the first organ such that said magnet may be positioned within the first organ and magnetically attract the receptacle inflated with said magnetic material thereto and thereby draw a portion of the second organ containing the inflated receptacle toward a corresponding portion of the first organ into sealing engagement therewith.

15. A surgical instrument according to claim 14 further comprising a second lumen in said catheter assembly for receiving a guide member therethrough.

16. A surgical instrument according to claim 14 wherein said magnetic material comprises material selected from the group of materials consisting of magnetic beads, magnetic pellets, magnetic particles, and magnetic dust.

17. A surgical instrument according to claim 16 wherein said pressure medium comprises air or a fluid solution.

18. A surgical instrument according to claim 14 further comprising a luer fitting in fluid communication with said supply lumen.

19. A surgical instrument according to claim 14 wherein said proximal end portion of said catheter assembly is removably coupled to said distal end portion of said catheter assembly.

20. A method for processing an instrument for surgery, the method comprising:
obtaining the surgical instrument of claim 14;
sterilizing the surgical instrument; and
storing the instrument in a sterile container.
